(19) United States
(12) Patent Application Publication
(10) Pub. No.: US 2006/0100176 A1
(43) Pub. Date: May 11, 2006

(54) Carboxylic acid diesters, methods for the production thereof and methods for the production of pharmaceutical active substances coupled to free amino groups with polysaccharide or polysaccharide derivatives

(76) Inventor: Klaus Sommermeyer, Rosbach (DE)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 10/542,944

(22) PCT Filed: Jan. 22, 2004
(86) PCT No.: PCT/EP04/00488

§ 371 Date: Jul. 20, 2005

(30) Foreign Application Priority Data
Jan. 23, 2003 (DE) 103 02 520.0

(51) Int. Cl. A61K 31/717; C08B 31/02; C08B 33/02

(52) U.S. Cl. 514/60; 536/48; 536/110

(57) ABSTRACT

The invention relates to carboxylic acid diesters of starch fractions or starch fraction derivatives in addition to solids and solutions containing said carboxylic acid diesters. The invention also relates to methods for the production of said carboxylic acid diesters, methods for the production of pharmaceutical active substances coupled to free amino functions with polysaccharides or polysaccharide derivatives and pharmaceutical substances thus obtained.

The present invention relates to carboxylic acid diesters, solids and solutions which comprise these esters and also to methods for their production. In addition, the present invention relates to methods for the production of pharmaceutical active substances coupled at free amino groups to polysaccharides or polysaccharide derivatives, which methods are carried out using the carboxylic acid diesters, and also to the pharmaceutical active substances which are obtainable by these methods.

The conjugation of pharmaceutical active substances, in particular proteins, to polyethylene glycol derivatives (“PEGylation”), or polysaccharides such as dextrans, or in particular hydroxyethyl starch (“HESylation”) has become of importance in recent years with the increase in pharmaceutical proteins from biotechnological research.

Frequently, such proteins have too short a biological half life which can be prolonged in a targeted manner by coupling to the abovementioned polymer compounds such as PEG or HES. By means of the coupling, however, the antigenic properties of proteins can also be beneficially affected. In the case of other pharmaceutical active compounds, by means of the coupling, the water solubility can be considerably increased.

DE 196 28 705 and DE 101 29 369 describe methods, such as coupling to hydroxyethyl starch in anhydrous dimethyl sulfoxide (DMSO), via which the corresponding aldonic acid lactone of the hydroxyethyl starch can be carried out using free amino groups of hemoglobin or amphotericin B.

Since, precisely in the case of proteins, anhydrous aprotic solvents can frequently not be employed, either for solubility reasons, or else reasons of protein denaturation, coupling methods using HES in an anhydrous environment are also available. For example, coupling of the reducing chain ends selectively to the aldonic-acid-oxidized hydroxyethyl starch succeeds via mediation of water-soluble carbodiimide EDC (1-ethyl-3-(3-dimethylaminopropyl)carbodiimide) (PCT/EP 02/02928). However, the use of carbodiimides is very frequently burdened with disadvantages, since carbodiimides very frequently cause inter- or intramolecular crosslinking reactions of the proteins as side reactions.

In the case of phosphate-containing compounds such as nucleic acids, the coupling often does not succeed at all, since the phosphate groups can likewise react with EDC (S. S. Wong, Chemistry of Protein Conjugation and Cross-Linking, CRC-Press, Boca Raton, London, New York, Washington D.C., 1993, page 199).

In consideration of the discussed prior art, the object underlying the invention was to provide compounds which, avoiding the above described disadvantages, make possible in a targeted manner the coupling of polysaccharides or their derivatives to amino-containing active substances, in particular proteins, in purely aqueous systems, or else in a solvent mixture with water.

Furthermore, such a compound should be of a nature such that binding as quantitative as possible of an active substance takes place due to covalent bonding to a polysaccharide or a polysaccharide derivative.

The object further underlying the invention was to provide compounds which make possible a linkage as mild as possible from a polysaccharide or a derivative thereof to an active substance. For instance, in particular the structure, the activity and the compatibility of the active substance should be changed as little as possible by the reaction. For example, intra- and intermolecular crosslinking reactions should be avoided. Furthermore, active substances which have phosphate groups should also be able to be linked.

Furthermore it was consequently an object of the present invention to specify compounds to which active substances could be coupled in a predetermined amount. For instance, in particular a targeted stoichiometry of the conjugate should be able to be established, in which case, especially, the production of conjugates should be made possible by the use of these compounds, which conjugates have a high proportion of active substance.

Finally, the object underlying the invention was to provide a method as simple and inexpensive as possible for producing such compounds and coupling products of polysaccharides or polysaccharide derivatives to active substances.

These objects are achieved, and also other objects which, although they are not mentioned directly, they can be derived as obvious from the context discussed herein, or inevitably result from these, using the carboxylic acid diesters described in claim 1. Expedient modifications of these inventive carboxylic acid diesters and also carboxylic acid diesters which are long-lasting and usable in methods for producing conjugates are claimed in the subclaims 2-19 which refer back to claim 1.

With respect to a method for producing carboxylic acid diesters, claims 20-24 provide a solution of the underlying object.

Claims 25-30 describe methods for producing polysaccharide-active substance conjugates and the pharmaceutical active substances obtainable by these methods.

By providing carboxylic acid diesters which are derived from polysaccharides or polysaccharide derivatives, it is possible to provide compounds which achieve the abovementioned objects. In the aqueous environment, they react with nucleophilic NH₂ groups to form urethanes.

In addition, by means of the present invention, inter alia, the following advantages are achieved:

The inventive carboxylic acid diesters make possible easy binding of an active substance by covalent bonding to a polysaccharide or a polysaccharide derivative.

The carboxylic acid diesters of the present invention can be reacted under mild conditions with an active substance. In this case, in particular the structure, the activity and the compatibility of the active substance is changed only to a slight extent by the reaction. By this means, inter alia, in particular intra- and intermolecular crosslinking reactions can be avoided. Furthermore, pharmaceutical active substances which have phosphate groups can be coupled, without these groups being changed.

The inventive carboxylic acid diesters permit a very gentle coupling to the active substance. Furthermore, for example a targeted stoichiometry of the desired conjugate can be set, in which case especially the production of conjugates is made possible by the use of these compounds, which conjugates have a high proportion of active substances.

Moreover, the present invention provides simple and inexpensive methods for producing activated carboxylic acid diesters and coupling products of polysaccharides or polysaccharide derivatives to active substances.

The carboxylic acid diesters of the present invention are derived from polysaccharides or polysaccharide derivatives. Such polysaccharides, and also derivatives obtainable therefrom, are widely known in the specialist area and can be obtained commercially. Polysaccharides are macromolecular carbohydrates, the molecules of which have a great number (minimum>10, but usually considerably more) of monosaccharide molecules (glycose) which are glycosidically linked to one another. The weight-average molecular weight of preferred polysaccharides is preferably in the range from 1500 to 1 000 000 dalton, particularly preferably 2000 to 300 000 dalton, and very particularly preferably in the range from 2000 to 50 000 dalton. The molecular weight Mw can be determined by customary methods. These comprise, for example, aqueous GPC, HPLC, light scattering and the like.

Via the molecular weight of the polysaccharide radical, inter alia, the residence time in the body can be changed.

The preferred polysaccharides comprise starch and also the starch fractions obtainable by hydrolysis which can be summarized as starch breakdown products. Starch is customarily subdivided into amylose and amylopectin, which differ in the degree of branching. According to the invention, amylopectin is preferred.

Amylopectins are taken to mean first quite generally branched starches or starch products having α-(1-4) and α-(1-6) bonds between the glucose molecules. The chains are branched in this case by the α-(1-6) bonds. These, in the case of naturally occurring amylopectins, are present irregularly about every 15-30 glucose segments. The molecular weight of natural amylopectin is very high in the range from 10⁷ up to 2×10⁸ dalton. It is assumed that amylopectin also forms helices to a certain extent.

A degree of branching can be defined for amylopectins. The index of the branching is the ratio of the number of molecules of anhydroglucose which bear branching points (α-(1-6) bonds) to the total number of molecules of anhydroglucose of the amylopectin, this ratio being expressed in mol-%. Amylopectin occurring naturally has degrees of branching of approximately 4 mol %. Amylopectins preferably used for producing the carboxylic acid diesters have a mean branching in the range from 5 to 10 mol %.

In addition, hyper-branched amylopectins can be used which have a degree of branching significantly exceeding the degree of branching known from nature for amylopectins. The degree of branching is in any case a mean value (mean degree of branching), since amylopectins are polydisperse substances.

Such hyper-branched amylopectins have significantly higher degrees of branching, expressed as mol % of the branching anhydroglucoses, compared with unmodified amylopectin or hydroxyethyl starch and are therefore more similar in their structure to glycogen.

The mean degree of branching of the hyper-branched amylopectins is customarily in the range between >10 and 25 mol %. This means that these amylopectins have, on average, about every 10 to 4 glucose units one α-(1-6) bond, and thus a branching point.

An amylopectin type which is preferably usable in the medical field is characterized by a degree of branching between 11 and 16 mol %.

Further preferred hyper-branched amylopectins have a degree of branching in the range between 13 and 16 mol %.

The amylopectins which are usable in the invention preferably have a value of the weight average molecular weight Mw in the range from 2000 to 800 000 dalton, in particular 2000 to 300 000, and particularly preferably 2000 to 50 000 dalton.

The starches described above can be obtained commercially. Furthermore, their production is known from the literature. For instance, starch, in particular from potatoes, tapioca, manioc, rice, wheat or corn can be produced. The starches obtained from these plants are frequently first subjected to a hydrolytic breakdown reaction. In this reaction the molecular weight is reduced from about 20 000 000 dalton to several million daltons, a further breakdown of the molecular weight to the previously mentioned values likewise being known. Particularly preferably, inter alia waxy corn starch breakdown fractions can be used for producing the inventive carboxylic acid diesters.

The above-described hyper-branched starch fractions are described, inter alia in German patent application 102 17 994.

In addition, derivatives of polysaccharides can also be used for producing the inventive carboxylic acid diesters. These comprise, in particular hydroxyalkyl starches, for example hydroxyethyl starch and hydroxypropyl starch, which can be obtained by hydroxyalkylation from the starches described above, in particular from amylopectin. Of these, hydroxyethyl starch (HES) is preferred.

Preferably, according to the invention an HES is used which is the hydroxyethylated derivative of the glucose polymer present at more than 95% in waxy corn starch, amylopectin. Amylopectin consists of glucose units which are present in α-1,4-glycosidic bonds and have α-1,6-glycosidic branches.

HES has advantageous rheological properties and is currently clinically used as volume-replacement agent and for hemodilution therapy (Sommermeyer et al., Krankenhauspharmazie, Vol. 8 (8, 1987) pages 271-278 and Weidler et al., Arzneimittelforschung/Drug Res., 41, (1991) pages 494-498).

HES is essentially characterized via the weight-average mean molecular weight Mw, the number average of the mean molecular weight Mn, the molecular weight distribution and the degree of substitution. Substitution with hydroxyethyl groups in the ether bond is possible here at the carbon atoms 2, 3 and 6 of the anhydroglucose units. The degree of substitution can be described here as DS (“degree of substitution”), which relates to the proportion of substituted glucose molecules of all glucose units, or as MS (“molar substitution”), which describes the mean number of hydroxyethyl groups per glucose unit.

The degree of substitution MS (molar substitution) is defined as the mean number of hydroxyethyl groups per anhydroglucose unit. It is determined from the total number of hydroxyethyl groups in a sample, for example according to Morgan, by ether cleavage and subsequent quantitative determination of ethyl iodide and ethylene which are formed in this case.

On the other hand, the degree of substitution DS is defined as the proportion of the substituted anhydroglucose units of all anhydroglucose units. It can be determined from the measured amount of unsubstituted glucose after hydrolysis of a sample. From these definitions, the fact that MS>DS results. In the event that only monosubstitution is present, that is to say every substituted anhydroglucose unit bears only one hydroxyethyl group, MS=DS.

A hydroxyethyl starch radical preferably has a degree of substitution MS of 0.1 to 0.8. Particularly preferably, the hydroxyethyl starch radical has a degree of substitution MS of 0.4 to 0.7.

The reactivity of the individual hydroxyl groups in the unsubstituted anhydroglucose unit toward hydroxyethylation differs depending on reaction conditions. Within broad limits, as a result the substitution pattern, that is to say the individual differently substituted anhydroglucoses which are randomly distributed on the individual polymer molecules, can be influenced. Advantageously, the C₂ and C₆ positions are predominantly hydroxyethylated, with the C₆ position, owing to its easier accessibility, being more frequently substituted.

Preferably, use is made in the context of this invention of hydroxyethyl starches (HES) which are predominantly substituted in the C₂ position, which starches are substituted as homogeneously as possible. The production of such HESs is described in EP 0 402 724 B2. They can be broken down without residue within a physiologically acceptable time and, on the other hand, nevertheless have a controllable elimination behavior. The predominant C₂ substitution makes the hydroxyethyl starch relatively poorly degradable for α-amylase. It is advantageous that, as far as possible, within the polymer molecules, no successively substituted anhydroglucose units occur, in order to ensure degradability without residue. In addition, such hydroxyethyl starches, despite the low substitution, have a sufficiently high solubility in the aqueous medium, so that the solutions are stable even over relatively long periods of time, and do not form agglomerates or gels.

Based on the hydroxyethyl groups of the anhydroglucose units, a hydroxyethyl starch radical preferably has a ratio of C₂:C₆ substitution in the range from 2 to 15. Particularly preferably, the ratio of C₂:C₆ substitution is 3 to 11.

In addition to the polysaccharide, the inventive carboxylic acid diesters comprise a further group derived from an alcohol. The term alcohol comprises compounds which have HO groups, with preferred alcohols differing from the polysaccharides or their derivatives. The HO groups can, inter alia, be bound to a nitrogen atom or to a phenyl radical.

Preferably, azide alcohols are used which are known in the specialist field. These comprise, inter alia, N-hydroxyimides, for example N-hydroxysuccinimide and sulfo-N-hydroxysuccinimide, substituted phenols and hydroxyazoles, for example hydroxybenzotriazole, with N-hydroxysuccinimides and sulfo-N-hydroxysuccinimide being particularly preferred.

Further suitable azide alcohols for producing the inventive carboxylic acid diesters are listed in the literature (V. H. L. Lee. Ed. Peptide and Protein Drug Delivery, Marcel Dekker, 1991, p. 65).

According to a particular aspect of the present invention, use is made of alcohols, the HO group of which has a pKₐ in the range from 6 to 12, preferably in the range from 7 to 11. This value is based on the acid dissociation constant determined at 25° C., this value being stated many times in the literature.

The molecular weight of the alcohol is preferably in the range from 80 to 500 g/mol, in particular 100 to 200 g/mol.

The inventive carboxylic acid diesters can be prepared via methods which are known per se. According to a particular aspect of the present invention, to prepare the inventive compounds, use is made of carboxylic acid diesters, the alcohol components of which differ from the polysaccharides or their derivatives. These compounds enable a particularly rapid and mild reaction, in which only alcohols and the desired carboxylic acid diesters are formed.

Preferred carboxylic acid diesters are, inter alia, N,N-succinimidyl carbonate and sulfo-N,N-succinimidyl carbonate.

These carboxylic acid diesters can be used in relatively small amounts. For instance, the carboxylic acid diester can be used in 1 to 3-molar excess, preferably 1 to 1.5-molar excess, based on the polysaccharide and/or the polysaccharide derivative. The reaction period when carboxylic acid diesters are used is relatively small. For instance, the reaction can frequently be terminated after 2 hours, preferably after 1 hour.

Depending on the desired stoichiometry, larger amounts can also be used. According to a particular aspect of the present invention, the ratio of carboxylic acid diesters to polysaccharide and/or polysaccharide derivative in the reaction is in the range of greater than 3:1 to 30:1, preferably 4:1 to 10:1.

The reaction to give the inventive carboxylic acid diester preferably takes place in an anhydrous aprotic solvent. The water content should preferably be at most 0.5% by weight, particularly preferably at most 0.1% by weight. Suitable solvents are, inter alia, dimethyl sulfoxide (DMSO), N-methylpyrrolidone, dimethylacetamide (DMA) and/or dimethylformamide (DMF).

The reaction to give the carboxylic acid diester succeeds under mild conditions. For instance, the above-described reactions can be carried out at temperatures preferably in the range from 0° C. to 40° C., particularly preferably 10° C. to 30° C.

According to a particular aspect of the present invention, the reaction takes place at a low base activity. The low base activity can be measured by adding the reaction mixture in a 10-fold excess. Here, the water, before addition, has a pH of 7.0 at 25° C., with the water comprising essentially no buffer. By measuring the pH at 25° C. after addition of the reaction mixture, the base activity of the reaction mixture is obtained. Preferably, this mixture, after addition, has a pH of at most 9.0, particularly preferably at most 8.0, and particularly preferably at most 7.5.

The solutions obtained by the above-described reaction can be used in the coupling reactions without isolation of the carboxylic acid diesters. Since, generally, the volume of the pre-activated carboxylic acid diesters in the aprotic solvent is low, compared with the target protein dissolved in the buffer volume, the amounts of aprotic solvent generally do not interfere. Preferred solutions comprise at least 10% by weight of carboxylic acid diesters, preferably at least 30% by weight of carboxylic acid diesters, and particularly preferably at least 50% by weight of carboxylic acid diesters.

The carboxylic acid diesters can be precipitated from the solution in aprotic solvent, for example DMF, by known precipitants, for example anhydrous ethanol, isopropanol or acetone, and purified by multiple repetition of the process. Preferred solids comprise at least 10% by weight of carboxylic acid diesters, preferably at least 30% by weight of carboxylic acid diesters, and particularly preferably at least 50% by weight of carboxylic acid diesters.

Such carboxylic acid diesters can then, isolated solvent-free, be used for the coupling, for example for HESylation. In this case, then, no side reactions occur, as described above using EDC-activated acid.

Furthermore, for the coupling a solution of the activated carboxylic acid diesters of polysaccharides and/or polysaccharide derivatives can be added to an aqueous solution of the pharmaceutical active substance, which is preferably buffered, at a suitable pH. The pharmaceutical active substances comprise at least one amino group which can be reacted to give the urethane polysaccharides and/or polysaccharide derivatives. The preferred active substances comprise antibiotics, in particular amphotericin B, and also proteins and peptides.

The pH of the reaction depends on the properties of the active substance. Preferably if this is possible, the pH is in the range from 7 to 9, particularly preferably 7.5 to 8.5.

The coupling generally takes place at temperatures in the range from 0° C. to 40° C., preferably 10° C. to 30° C., without this being intended to be a restriction. The reaction period can be readily determined by suitable methods. Generally, the reaction time is in the range from 10 minutes to 100 hours, preferably 30 minutes to 5 hours.

The molar ratio of carboxylic acid diesters to active substance can lie in a wide range. Depending on the intended stoichiometry, the carboxylic acid diesters can be used in 1 to 5-fold molar excess, particularly preferably 1.5 to 2-fold excess, based on the pharmaceutical active substance. According to a further aspect of the present invention, the pharmaceutical active substance can be used in 2 to 20-fold molar excess, particularly preferably 3 to 10-fold excess, based on the carboxylic acid diesters.

As by-product in the abovementioned reaction, essentially only the alcohol occurs, for example N-hydroxysuccinimide, which can be readily separated off from the coupling product, e.g. by ultrafiltration.

As a side reaction, a saponification of the carboxylic acid diesters with water can occur, in which case the polysaccharides and/or polysaccharide derivatives used, free alcohol and also CO₂ are formed. It is particularly surprising, therefore, that the inventive carboxylic acid diesters, for the most part, undergo a coupling reaction with a pharmaceutical active substance. This follows from the examples.

The invention will be described in more detail below by examples and comparative examples, without the invention being intended to be restricted to these examples.

### EXAMPLES AND PRODUCTION METHODS

### Example 1

### Production of HES 10/0.4-carboxylic acid diester of N-hydroxysuccinimide

5 g of dried hydroxyethyl starch having a mean molecular weight Mw=10 000 dalton and a degree of substitution MS=0.4 are dissolved in 30 ml of anhydrous dimethylformamide at 40° C. and, after cooling the solution, are admixed with the equimolar amount of N,N′-disuccinimidyl carbonate with exclusion of moisture. After stirring for 2 hours at room temperature, the carboxylic acid diester of the N-hydroxysuccinimide and HES which is formed is directly further processed as described in example 2.

### Example 2

### Production of HES 10/0.4-coupled myoglobin

5 mg of myoglobin are dissolved in 0.4 ml of bicarbonate buffer, 0.3 molar pH 8.4. To the solution is added 0.5 ml of the solution from example 1 containing the HES 10/0.4-carboxylic acid diester of N-hydroxysuccinimide at room temperature in portions over 2 hours. The batch is stirred for 1 hour. The formation of the HESylated myoglobin is determined via gel permeation chromatography at a yield of >90%, based on the myoglobin used.

### Example 3

### Production of HES 10/0.4-coupled amphotericin B

100 mg of amphotericin B are dissolved in 5 ml of anhydrous DMSO under protective gas treatment with argon under protection from light.

To this solution is added a solution of HES 10/0.4-carboxylic acid diester of N-hydroxysuccinimide produced according to example 1 and produced using double the molar amount of N,N′-disuccinimidyl carbonate, and the mixture is allowed to react to completion at room temperature for 4 hours under argon and protection from light.

The batch is then diluted with 200 ml of oxygen-free water under argon and ultrafiltered under protection from light and argon using a membrane having a cutoff of 1000 dalton for removing the solvent and the N-hydroxysuccinimide liberated.

The batch is then freeze-dried for isolation of the reaction product. The product is characterized via gel chromatography and photometric determination of the proportion of coupled amphotericin B via photometry.

Yield based on amphotericin B used, 90%. The molecular weight determined was 12 000 dalton and the proportion of the coupled amphotericin B approximately 20%, equivalent to a molar ratio of 2:1.

1-30. (canceled)

31. An aprotic-solvent-soluble carboxylic acid diester of polysaccharides or polysaccharide derivatives having a mean content of no more than 10 carboxylic acid diester substituents per polysaccharide molecule.

32. The carboxylic acid diester as claimed in claim 31, characterized in that the polysaccharides or polysaccharide derivatives are starch fractions or starch fraction derivatives.

33. The carboxylic acid diester as claimed in claim 32, characterized in that the starch fractions are breakdown fractions of amylopectin.

34. The carboxylic acid diester as claimed in claim 33, characterized in that the breakdown fractions of amylopectin are obtained by acid breakdown and/or breakdown by α-amylase of waxy corn starch.

35. The carboxylic acid diester as claimed in claim 34, characterized in that the starch fractions have a mean molecular weight Mw of 2000-50 000 dalton and a mean branching of 5-10 mol % of α-1,6-glycosidic bonds.

36. The carboxylic acid diester as claimed in claim 34, characterized in that the starch fractions have a mean molecular weight Mw of 2000-50 000 dalton and a mean branching in the range from >10 to 25% of α-1,6-glycosidic bonds.

37. The carboxylic acid diester as claimed in claim 32, characterized in that the starch fraction derivatives are hydroxyethyl derivatives of breakdown fractions of waxy corn starch.

38. The carboxylic acid diester as claimed in claim 37, characterized in that the mean molecular weight of Mw of the hydroxyethyl starch fractions is in the range 2-300 000 dalton and the degree of substitution Ms is between 0.1 and 0.8, and also the C2/C6 ratio of the substituents on the carbon atoms C2 and C6 of the anhydroglucoses is between 2 and 15.

39. The carboxylic acid diester as claimed in claim 31, characterized in that an alcohol from which the alcohol component of the carboxylic acid diester is derived has a molecular weight in the range from 80 to 500 g/mol.

40. The carboxylic acid diester as claimed in claim 31, characterized in that an alcohol from which an alcohol component of the carboxylic acid diester is derived has a pKₐ in the range from 6 to 12.

41. The carboxylic acid diester as claimed in claim 31, characterized in that an alcohol, from which an alcohol component of the carboxylic acid diester is derived, of the carboxylic acid diester comprises an HO—N group or a phenol group.

42. The carboxylic acid diester as claimed in claim 31, characterized in that an alcohol from which the alcohol component of the carboxylic acid diester is derived is selected from N-hydroxysuccinimide, sulfo-N-hydroxysuccinimide, substituted phenols and hydroxybenzotriazole.

43. The carboxylic acid diester as claimed in claim 42, characterized in that an alcohol from which an alcohol component of the carboxylic acid diester is derived is N-hydroxysuccinimide and sulfo-N-hydroxysuccinimide.

44. A solid comprising at least one carboxylic acid diester as claimed in claim 31.

45. A solution comprising at least one carboxylic acid diester as claimed in claim 31.

46. The solution as claimed in claim 45, characterized in that the solution comprises at least one organic solvent.

47. The solution as claimed in claim 46, characterized in that the solution comprises at most 0.5% by weight of water.

48. The solution as claimed in claim 45, characterized in that the solution comprises at least one aprotic solvent.

49. The solution as claimed in claim 48, characterized in that the solvent comprises dimethyl sulfoxide (DMSO), N-methylpyrrolidone, dimethylacetamide (DMS) and/or dimethylformamide (DMF).

50. A method for production of carboxylic diester as claimed in claim 31, characterized in that at least one polysaccharide and/or a polysaccharide derivative is reacted with at least one carboxylic acid diester in solution in aprotic solvent and the molar ratio of carboxylic ester to polysaccharide is not greater than 10:1.

51. The method as claimed in claim 50, characterized in that both alcohol components of the carboxylic acid diester have a PKₐ in the range 6 to 12.

52. The method as claimed in claim 51, characterized in the N,N′-disuccinimidyl carbonate is used as carboxylic acid diester.

53. The method as claimed in claim 50, characterized in that the reaction takes place at a temperature in the range from 0 to 40° C.

54. The method as claimed in claim 50, characterized in that the reaction takes place at a low base activity.

55. A method for producing pharmaceutical active substances coupled at free amino functions to polysaccharides or polysaccharide derivatives, characterized in that at least one carboxylic acid diester as claimed in claim 1 is reacted with a pharmaceutical active substance which has at least one amino group.

56. The method as claimed in claim 55, characterized in that the reaction takes place in aqueous medium.

57. The method as claimed in claim 56, characterized in that the pH of the aqueous medium is in the range from 7 to 9.

58. The method as claimed in claim 55, characterized in that the reaction takes place at a temperature in the range from 0° C. to 40° C.

59. The method as claimed in claim 55, characterized in that the pharmaceutical active substance is a polypeptide or a protein.

60. A pharmaceutically active substance obtained by a method as claimed in claim 25.
